(19) United States
(12) Patent Application Publication
(10) Pub. No.: US 2017/0046972 A1
(43) Pub. Date: Feb. 16, 2017

(54) Stop Clerk

(71) Applicant: Valerie Jean Whitcomb, Issaquah, WA (US)

(72) Inventor: Valerie Jean Whitcomb, Issaquah, WA (US)

(21) Appl. No.: 14/827,298

(22) Filed: Aug. 15, 2015

(51) Int. Cl. G09B 19/00; A61B 5/16

(52) U.S. Cl. CPC G09B 19/00; A61B 5/165

(57) ABSTRACT

A portable/wearable self-help invention providing the user with a neutral action and timed interval for a pause to choose a response instead of reacting without thought. The invention provides a set of buttons to assist with choosing responses to intense emotions, to consolidating good memories, and to cancelling criticism. The invention does not change a person's thinking; it provides them an opportunity to choose changes in their thinking. The features are leverage brain science principles.

The invention provides feedback to the user, an action to take, and a scientifically-based time interval to accomplish specific goals to improve the user's thought life and therefore the ability to choose responses beyond habits and reflex.

### RELATED U.S. PATENT DOCUMENTS

My search for thought-assistance devices revealed talk therapy sessions and devices to alert emergency responders but no devices to assist people in pausing and examine their thinking.

### BACKGROUND-FIELD OF THE INVENTION

The present disclosure relates to a device and methods for an individual to make intentional choices to promoting reasoned responses rather than habitual/reflexive reaction to circumstances.

Today's news is filled with stories of people acting under the influence of uncontrolled emotions and making life-changing mistakes impacting themselves and others. The rapid pace of our society has taxed the capacity of our physiology to naturally handle the stimuli and stressors of our present-day world. For some, the emotional burden is overwhelming and they need assistance regaining balance in their thinking, that is, mindfulness. For example, people suffering from Post -Traumatic Stress need to regain their sense of control in order to overcome the psychological changes the event or series of events caused in their lives. The invention provides a set of buttons to assist with choosing responses to intense emotions, consolidating good memories, and cancelling criticism. The invention does not change a person's thinking; it gives them an opportunity to choose changes in their thinking

### DETAIL DESCRIPTION

This patent application describes a specialized use for a set of software-configurable buttons on a portable personal device designed to help people choose intentional responses and extinguish reflexive reactions. One method includes alerting an individual to thoughtfully respond to intense emotions. Another method alerts an individual to store good memories for easier recall later and promotes a sense of well-being. A third method promotes mindful consideration of constructive criticism/correction.

Feature 1: Choose To Pause To Master Intense Emotion

Studies have shown our bodies react to triggering events by dumping chemicals, clouding our decision making ability. The invention provides a practical implementation of what is called the 90 Second Rule to get control. This rule is from the book, My Stroke of Insight, by Jill Bolte Taylor in which she states, “When a person has a reaction to something in their environment, there's a 90 second chemical process that happens in the body; after that, any remaining emotional response is just the person choosing to stay in that emotional loop. Something happens in the external world and chemicals are flushed through your body which puts it on full alert. For those chemicals to totally flush out of the body it takes less than 90 seconds. This means that for 90 seconds you can watch the process happening, you can feel it happening, and then you can watch it go away. After that, if you continue to feel fear, anger, and so on, you need to look at the thoughts that you're thinking that are re-stimulating the circuitry that is resulting in you having this physiological response over and over again.”

Other supporting principles for the efficacy of the device are noted by Michele Rosenthal, Trauma Coach: “One of the toughest actions for trauma survivors is to learn how to use their mind to control their mind. Sometimes, the easiest way to do that is by creating habits that facilitate this process from an external source. Having a device that will countdown the seconds to more mental clarity offers users (1) an activity to replace the intense emotion, (2) a diversion to habitual thoughts and replacement with a task, (3) teachable moment for how to pause and reclaim control.” This invention provides the user with all three benefits should the user choose control. 1. An action, 2. A diversion, and 3. A timed pause that matching what science suggests.

Feature 2: Choose To Consolidate a Positive Memory

Brain studies also support the idea that one must pause and intentionally choose to consolidate an emotional memory. The pause can be between 5-30 seconds. The invention will include a 30 second timer with feedback to assist the user in acting on the intent to remember something good and pausing for a time to consolidate the memory.

Supporting information from Michele Rosenthal: “The 30 seconds I adopted from Rick Hanson [a Neuropsychologist] and his discussion about ‘taking in the good’: “Positive experiences generally have to be held in awareness for 5-10-20 seconds for them to register in emotional memory.” I promote 30 seconds because I think the reinforcement of the focus and experience helps to deepen the effects. You can see Hanson's statement here:

http://media.rickhanson.net/home/files/TakingintheGood.pdf.”

Feature 3: Cancel a Negative Thought

We all know that not every critical thought we have is wholly accurate. Some thoughts are only destructive without any instructive value. However, they are often the thoughts we tend to dwell on to our own detriment. A third feature of the invention would allow the user to intentionally take an action to reinforce a decision to discard destructive thoughts and criticism—a cancel feature. The rationale for this feature is supported by the teaching of Laura King, a hypnotherapist, who advocates using the word “cancel” as a trigger to counteract and stop further contemplation of unhelpful remarks or thinking This feature is not a timer like the others, but a feature offering the user an action to take to reinforce the concept of cancelling the negative. This feature provides feedback to confirm the user's action.

### SUMMARY

In accordance with the invention, the Stop Clerk (formerly RESPONSE ASSISTANT) incorporates a portable/wearable shell, a miniature circuit card, a set of buttons (1-4), and feedback elements.

### BRIEF DESCRIPTION OF THE DRAWING

FIG. 1: A key fob-like general arrangement with four buttons

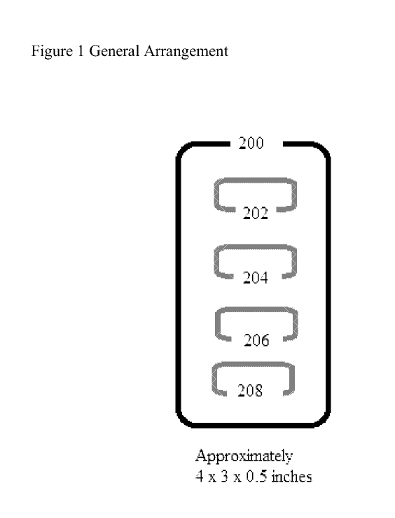

### CONCLUSION, RAMIFICATION, AND SCOPE

While the above description contains many specificities, these should not be construed as limitations on the scope of the invention, but as exemplifications of the presently preferred embodiments thereof. Many ramifications and variations are possible via the utility of the software configurable time intervals and the invention's embodiment. For example, it could be packaged as a wearable art piece of various fashion designs. Thus the scope of the inventions utility should be determined by any appended claims and their legal equivalents, and not by the examples given, e.g. Post Traumatic Stress.

The Stop Clerk (formerly RESPONSE ASSISTANT) invention is a self-help tool designed to give the user an action to take and a scientifically-based time interval to accomplish specific goals to improve the' user's thought life and therefore the ability to choose responses beyond habits and reflex.

After reviewing a presentation for the Stop Clerk (formerly RESPONSE ASSISTANT), Pia Jansen, MSW, EAGALA: Advanced- certified, endorsed the invention. “I've been a therapist for more than 30 years. In our work, I find that most of my clients would benefit from what I call the ‘Power of Pause.’ The RESPONSE ASSISTANT [renamed Stop Clerk] can help people learn to use this power. The possibilities are endless in regards to healing from all sorts of life obstacles! I fully support this idea. I highly recommend prototyping and testing. I look forward to using the Stop Clerk (formerly RESPONSE ASSISTANT) in my practice.”

What is claimed is:

1. An objective of the portable/wearable invention is to provide an individual with convenient, repeatable access to known neuroscientific principles for application in their immediate circumstance.
The preferred embodiment of the device is a set of buttons and feedback features that may or may not be part of a button but do provide information to the user. To accommodate future expansion of functionality, I have included a fourth button of whose function is To Be Assigned (TBA).
The Stop Clerk (formerly RESPONSE ASSISTANT) device is a self-help invention designed to provide the user with an action to take and a scientifically-based time interval to accomplish specific goals to improve the user's thought life and therefore the ability to choose responses beyond habits and reflex. The device promotes conscious involvement on the part of the individual. The intended use drives the design.
#1 The embodiment is a portable device with buttons which in the case of features 1 and 2, provide a short vibration to indicate activation and a longer vibration to indicate the end of the timed interval.
#2 Buttons the user presses to initiate an operation. The invention provides feedback (vibration or sound) to confirm selection and after a set time feedback (vibration or sound) to indicate the end of the programmed time interval.
#3 A physical invention with a logical purpose but no specifics of the internal implementation to allow flexibility in engineering the prototype.
